(19) United States
(12) Patent Application Publication
(10) Pub. No.: US 2012/0065279 A1
(43) Pub. Date: Mar. 15, 2012

(54) BIMETALLIC MO/CO CATALYST FOR PRODUCING OF ALCOHOLS FROM HYDROGEN AND CARBON MONOXIDE CONTAINING GAS

(75) Inventor: Caili Su, Burnaby (CA)

(73) Assignee: SYNTHENOL ENERGY CORPORATION, Vancouver (CA)

(21) Appl. No.: 13/002,164

(22) PCT Filed: Jun. 22, 2009
(86) PCT No.: PCT/US09/48132

§ 371 Date: Oct. 31, 2011

Related U.S. Application Data
(60) Provisional application No. 61/078,042, filed on Jul. 3, 2008.

(51) Int. Cl. C07C 27/06; B01J 23/75; B01J 23/28

(52) U.S. Cl. 518/714; 502/306; 502/313; 502/174

(57) ABSTRACT

Carried catalysts for producing alcohols from gaseous mixtures containing hydrogen and carbon monoxide, e.g., syngas, are made from precursors of a particulate inert porous catalyst substrate impregnated with the oxides or salts of molybdenum, cobalt, and a promoter alkali or alkaline earth metal, in a molybdenum to cobalt molar ratio of from about 2:1 to about 1:1, preferably about 1.5:1, and in a cobalt to alkali metal molar ratio of from about 1:0.08 to about 1:0.30, preferably about 1:0.26-0.28. The catalysts are “activated” by reducing the catalyst precursor material in a reducing environment at from about 600° C. to about 900° C., preferably about 800° C. Alcohols are produced by passing gas mixtures containing at least CO and H₂ in ratios of from 1:1 to 3:1 through a reactor containing the catalyst, at from about 240° C. to about 270° C., and a pressure of 1000-1200 psi.

### CROSS-REFERENCE TO RELATED APPLICATIONS

This application claims priority of U.S. Provisional Patent Application Ser. No. 61/078,042 filed Jul. 3, 2008.

### FIELD AND BACKGROUND

The present invention relates to the field of catalysts which are especially useful in facilitating the reactions of gaseous ingredients such as CO and H₂, to ultimately form alcohols, and to their preparation and use. U.S. Pat. Nos. 4,825,013, 4,752,622, 4,882,360, 4,831,060, 4,752,623, 4,607,055, 4,607,056, and 4,661,525 are exemplary.

### SUMMARY OF THE INVENTION

The present invention encompasses carried catalyst precursors, carried catalysts, and methods of preparation of such catalysts, as well as producing alcohols from gaseous mixtures containing hydrogen and carbon monoxide, e.g. syngas, using the catalysts. The carried catalyst precursors comprise a particulate inert porous catalyst substrate carrying the oxides or salts of molybdenum, cobalt, and a promoter alkali or alkaline earth metal, in a molybdenum to cobalt molar ratio of from about 2:1 to about 1:1, preferably about 1.5:1, and in a cobalt to alkali metal molar ratio of from about 1:0.08 to about 1:0.30, preferably about 1:0.26-0.28.

The catalyst precursors are preferably formed by impregnating the porous catalyst substrate material with salts of molybdenum, cobalt and the promoter metal in the above indicated ratios, and calcining the carried salts to oxides, unless the salts used can be reduced without giving off products deleterious to the catalytic activity of the system, the reactor and/or the products of the catalyzed reaction.

The catalysts are formed, or “activated,” by reducing the catalyst precursor material in a reducing environment at from about 600° C. to about 900° C., preferably about 800° C.

Alcohols are produced by passing gas mixtures containing at least CO and H₂ through a reactor containing the catalyst, at from about 240° C. to about 270° C., and a pressure of 1000-1200 psi.

The H₂/CO ratio varies from 1:1 to 3:1, preferably about 1-1.5:1, and most preferably about 1:1. The yield of alcohols can reach 140-175 g/kg.cat h at a ratio of high alcohols (C₂⁺OH) to methanol about 0.9-1.0. If syngas is produced from a biomass gasification, which has a carbon efficiency of 67%, 115 gallon of alcohols can be produced from per bone dry ton of biomass, which is higher than the available fermentation processes.

These and other objects, features and advantages of the invention will be more fully understood and appreciated by reference to the Description of the Preferred Embodiments below.

### DESCRIPTION OF THE PREFERRED EMBODIMENTS THE CATALYST

Catalyst Precursor Preparation

In the preferred embodiment, salts of molybdenum, cobalt and an alkali or alkaline earth metal promoter are sequentially loaded onto a porous inert substrate material. Ammonium molybdenate tetrahydrate is a preferred molybdenum salt. Cobalt nitrate is a preferred cobalt salt. The most preferred promoter is cesium, and cesium formate is a preferred cesium salt.

Exemplary porous inert materials suitable as catalyst substrates include powdered, granular or otherwise particularized carbon, titanium dioxide, zirconium dioxide and alumina. A presently preferred substrate is alumina (Al₂O₃), preferably in spherical particle form, having a particle size of from about 1.5 to about 2.0 millimeters (mean diameter), preferably about 1.8 millimeters, a density of about 0.63 grams per cubic millimeter, a surface area of about 210 m² per gram, and a pore volume of about 0.75 cubic millimeters per gram.

The molar ratio of molybdenum to cobalt to promoter metal used in catalyst is about:

1-2:1:0.08-0.30, preferably about 1.5:1:0.26-0.28.

When alumina is used as the substrate, from about 5.7 to about 11.4 wt % Mo (based on weight of Mo to Al₂O₃) is loaded onto and to some extent impregnated into the substrate. In other words, from about 5.7 to about 11.4 grams of molybdenum is loaded per 100 grams of substrate. Preferably from 8.5-10 wt % molybdenum is loaded onto the substrate. The other salts loaded proportionally to obtain the above indicated molar ratios.

Each of the three metal salts is dissolved in its own aqueous solution. The required quantity of salt to be loaded onto the quantity of substrate used, is dissolved in a volume of water which approximately matches the volume of water which the amount of substrate used will absorb.

The substrate is preferably first impregnated with the ammonium molybdenate solution. It is dried at 60° C. for 4 hours, then overnight at 110° C. The cobalt nitrate solution is then applied and the substrate is dried in the same manner. After the molydenum and cobalt salts are impregnated into the substrate, the system is calcined at 350° C. for 4 hours in air. This converts the metal salts to oxides, which are subsequently activated by reduction in situ in the reactor, as indicated below.

Then the substrate and molybdenum-cobalt combination is impregnated with the cesium salt. The system is again dried in the same manner. The formate salt is an example of a salt which can be directly reduced without creating products which are deleterious to the catalyst and the reactor. This makes it unnecessary to calcine the cesium formate before catalyst activation, as the heat and reduction of activation will reduce the metal formate to the elemental metal, or to a metal hydride, with water and carbon dioxide being gassed off. The water and carbon dioxide do not foul the reactor/catalyst system or the alcohols produced in the catalyzed reaction.

Catalyst Precursor Activation

The catalyst precursor must be activated prior to use. The catalyst precursor-substrate combination is loaded into the reactor in which it will be used to produce alcohol. The catalyst precursor/substrate combination is heated in the reactor at about 600° C. to about 900° C., preferably about 800° C., at approximately atmospheric pressure, in a flowing stream of nitrogen and hydrogen in a 3/2 ratio by volume. This treatment is continued for about 3 to about 10 hours, preferably about 5 hours. The flow rate of the reducing gas mixture used is approximately 15 cc per minute per cc of catalyst precursor-substrate combination (15 cc/min/cc catalyst precursor-substrate). After this activation process, the catalyst is protected by using an inert gas environment before syngas is fed in to the reaction system.

Although not wishing to be bound by theory, it is believed that the cobalt oxide, molybdenum oxide and cesium formate are thereby reduced to elemental metals, and/or metal hydrides or alloys. Thus, the catalyst obtained comprises elemental molybdenum, cobalt or alloys and an alkali or alkaline earth metal, and/or hydrides thereof, in an elemental ratio of about 2-1:1:0.08-0.30, preferably about 1.5:1:0.26-0.28. It is carried on the porous, inert particularized material, such as alumina.

Once the catalyst activation is completed in this manner, the catalyst and reactor are ready for use.

### Reactor Operation

A gaseous mixture containing hydrogen and carbon monoxide is passed through the reactor under the operating conditions set forth below. In commercial gasification operation, a syngas mixture produced by thermal and generally anaerobic decomposition of a carbon containing mass in the presence of superheated steam will preferably be used. The ratio of hydrogen to carbon monoxide in the gaseous mixture is preferably about 1-1.5:1.

The reactor is operated at the relatively low temperature of from about 240 to about 270° C., preferably at a maximum of 260° C. Higher pressure is theoretically necessary, but low pressure is preferably employed considering the process cost, e.g. from about 1000 to about 1200 psig. The Gas Hourly Space Velocity (GHSV) used is from about 4000 to about 6000 h⁻¹. Lower temperature and higher pressure favor higher alcohol formation in this process.

### EXAMPLES

The following examples, set forth in Tables 1-6, show the results achieved by the catalysts of the present invention, and the effects of Co, Mo and Cs loading and ratios on the activity of the catalysts and reaction selectivities to alcohols. In all of the examples, the experiments were conducted based on a single pass of reactant containing gas through the reactor. None of the gas was recycled as would be done in a commercial operation.

The calculation of gallons of alcohol/BDT (Bone Dry Ton of Biomass) in the Tables was conducted as follows:
1. The moles of CO (A) introduced into the system during the testing time was measured.
2. The moles of CO (13) coming out of the reactor were measured.
3. The gallons of alcohol (G) produced in during the test period were measured.
4. G/[A−B] gives you the gallons of alcohol/mole of carbon (as CO) converted.
5. The assumption is made that in a commercial process, all of the carbon monoxide coming from a ton of biomass will eventually be converted through recycling of the gas through the reactor.
6. Then assuming, based on experience, that 667 pounds of carbon as CO will be produced from a gasified ton of dry biomass (BDT-bone dry ton), the ratio of G/[A−B] is used to calculate the gallons of alcohol which would result from that amount of carbon. This calculation assumes a theoretical efficiency of the gasifier to be 66.7% as one BDT of biomass (moisture and ash free) generally contains 1000 pounds of carbon.

“Con. % of CO” in the second column of the tables refers to the weight percent of CO which has been converted to other products in its pass through the reactor.

The “Selectivities of Alcohols C Mol %” in the third column refers to the mol % of carbons converted to the indicated alcohols.

### 1. Results Using Catalysts of the Same Formula

Test Catalyst: The catalyst used has Mo:Co:Cs ratios of 1:1:0.27. Mo was loaded onto the preferred alumina substrate at the 5.7 wt % (5.7 grams Mo per 100 grams alumina substrate).substrate

Test Conditions: Temp.: 265° C. Pressure: 1200 psi. GHSV: 4269-4321 h⁻¹ Syngas: CO/H₂=1:1

Test Time: The tests were conducted over a span of either 5 hours or 60 hours after the reaction became stable.

Condensers: #1, collected the liquid products of first 21 hours (of 60 hour-run)

#2, collected the liquid products of the last 13 hours (of 60 hour-run)

TABLE 1
Results summary for the 5-hours testing and 60-hours testing

| | Con. | | | | | | Alcohol | |
|---|---|---|---|---|---|---|---|---|
| Testing | % of | Selectivities of Alcohols C Mol % | | | | | Productivity · | G |
| Time/h | CO | MeOH | ETOH | 1-PrOH | 1-BuOH | Other-ROH | g/kgcat · h | Gallon/BDT |
| *5 | 4.7 | 15.3 | 15.8 | 5.0 | 1.6 | 2.2 | 89.1 | 89.1 |
| 21(#1) | 6.9 | 21.6 | 18.6 | 6.1 | 1.9 | 3.1 | 175.2 | 115.6 |
| 13(#2) | 5.0 | 17.2 | 13.6 | 4.4 | 1.4 | 2.1 | 94.9 | 87.8 |
| ** 5 | 6.0 | 17.0 | 14.4 | 4.7 | 1.4 | 2.6 | 120.9 | 90.1 |

*Same formulation of catalyst tested for 5 hours
** Same catalyst, tested for 5 hours after the 60-hours run

From the results in Table 1, it was shown that higher conversion and selectivity were obtained at the beginning 21 hours and then gradually the reaction became stable. When the reaction system was shut down and started again, both the conversion and selectivity (Row 5, Table 1) were able to reach as high as or higher than the prior levels. This indicated that the catalyst was not deactivated during the testing period.

Based on the above experimental results the average G value and alcohol productivity for the Table 1 results were:

The alcohol productivity: 119.9 g/kgcat.h

The G value: 95.6 Gallon/BDT

### 2. Effect of Co Loading on the Activity of Catalysts and Selectivities to Alcohols

Test Catalyst: The amount of Co used was varied, giving different Mo:Co ratios. Mo was loaded onto the preferred alumina substrate at 5.7 wt % and Cs was loaded at 2.2 wt %

Test Conditions: Temp.: 260-272° C. Pressure: 1200 Psi. GHSV: 4300 h⁻¹, except as indicated.

Syngas: CO/H₂=1:1

Test Time: 5 hours

TABLE 2
Effect of Co loading on the activity of catalysts and selectivities to alcohols

| Mo/Co | | | | | | | | |
|---|---|---|---|---|---|---|---|---|
| Loading | | | | | | | Alcohol | |
| Wt %/wt % | Con. % | Selectivities of Alcohols C Mol % | | | | | Productivity · | G |
| (molar ratio) | of CO | MeOH | ETOH | 1-PrOH | 1-BuOH | Other-ROH | g/kgcat · h | Gallon/BDT |
| *5.7/0 | 17.0 | 0.2 | 0.1 | — | — | — | 2.3 | — |
| 5.7/1.75 | 5.0 | 16.7 | 13.5 | 4.4 | 1.3 | 2.4 | 96.3 | 86.6 |
| (2:1) | | | | | | | | |
| 5.7/3.5 | 6.0 | 17.0 | 14.4 | 4.7 | 1.4 | 2.6 | 120.1 | 90.1 |
| (1:1) | | | | | | | | |
| 5.7/5.3 | 2.3 | 14.1 | 11.5 | 3.5 | 1.1 | 0.7 | 33.8 | 70.7 |
| (1:1.5) | | | | | | | | |
| #5.7/7.0 | 2.7 | 15.0 | 13.5 | 4.4 | 1.7 | 2.8 | 67.2 | 83.2 |
| (1:2) | | | | | | | | |

*Temp: 321° C., #239° C., GHSV: 5980 h⁻¹.
—Trace

The catalyst containing 5.7 wt % and Mo and Cs 2.2 wt % (without Co) was not active at all at the temperature of 260-270° C. It had 17% CO conversion at much higher temperature of 320° C., but only trace amount of alcohols was in the products. When the loading of Co was increased to 1.75 wt %, both the activity and the selectivity of alcohols were increased obviously. When the loading was increased to 3.5 wt %, the alcohol productivity reached 120 g/kgcat.h and the yield of alcohol (G value) reached 90 gallon/BDT. Both the activity and selectivity decreased when the loading of Co was increased to 5.3 wt %.

### 3. Effect of Mo Loading on the Activity of Catalysts and Selectivities to Alcohols

Test Catalyst: The amount of Mo used was varied, giving different Mo:Co ratios. Co was loaded onto the preferred alumina substrate at the 3.5 wt %; Cs was loaded at 2.2 wt %.

Test Conditions: Temp.: 241-255° C. Pressure: 1200 psi. GHSV: 5759-6000 h⁻¹, except as indicated.

Syngas: CO/H₂=1:1

Test Time: 5 hours

TABLE 3
Effect of Mo loading on the activity of catalysts and selectivities to alcohols

| Mo/Co | | | | | | | | |
|---|---|---|---|---|---|---|---|---|
| Loading | | | | | | | Alcohol | |
| Wt %/wt % | Con. % | Selectivities of Alcohols C Mol % | | | | | Productivity · | G |
| (molar ratio) | of CO | MeOH | ETOH | 1-PrOH | 1-BuOH | Other-ROH | g/kgcat · h | Gallon/BDT |
| *0/3.5 | — | — | — | — | — | — | 2.9 | — |
| 2.8/3.5 | 2.9 | 15.2 | 12.7 | 3.7 | 1.0 | 1.5 | 70.2 | 77.6 |
| (0.5:1) | | | | | | | | |
| 5.7/3.5 | 7.6 | 17.1 | 14.1 | 4.2 | 1.3 | 1.9 | 92.7 | 89.1 |
| (1:1) | | | | | | | | |
| 11.4/3.5 | 4.6 | 20.5 | 16.4 | 5.3 | 1.7 | 2.9 | 141.9 | 105.7 |
| (2:1) | | | | | | | | |
| 17.1/3.5 | 3.6 | 15.0 | 13.7 | 4.2 | 1.3 | 2.4 | 86.2 | 81.8 |
| (3:1) | | | | | | | | |

*GHSV: 4127 h⁻¹

Changing the loading of Mo wt % from 0-17.1%, the reaction selectivities and alcohol yield initially increased with increasing loading of Mo, and reached the highest level at a Mo loading of 11.4 wt %. Above about 11.4%, selectivities and yields decreased with continuing increase in Mo loading. The catalyst which does not contain Mo was not active either at the same temperature and pressure ranges, and even lower GHSV and higher temperature. Therefore, both Mo and Co, or their alloy, play a significant role for the catalyst to be active at the conditions applied.

### 4. Effect of Mo/Co Ratio on the Activity of Catalyst and Selectivity of the Reaction

Test Catalyst: The ratio of Mo to catalyst was varied. Cs was loaded at 2.2 wt % to the substrate.

Test Conditions: Temp.: 250° C. Pressure: 1200 psi. GHSV: 4330 h⁻¹, except as indicated.

Syngas: CO/H₂=1:1

Test Time: 5 hours

TABLE 4
Effect of Mo/Co ratio on the performance of the catalysts

| Mo/Co | | | | | | | | |
|---|---|---|---|---|---|---|---|---|
| Loading | | | | | | | Alcohol | |
| Wt %/wt % | Con. % | Selectivities of Alcohols C Mol % | | | | | Productivity · | G |
| (molar ratio) | of CO | MeOH | ETOH | 1-PrOH | 1-BuOH | Other-ROH | g/kgcat · h | Gallon/BDT |
| *2.8/1.75 | 3.6 | 9.9 | 9.5 | 2.7 | 0.6 | 1.0 | 45.3 | 53.8 |
| (1:1) | | | | | | | | |
| 5.7/1.75 | 5.4 | 15.9 | 14.1 | 4.1 | 1.2 | 2.3 | 101.8 | 84.7 |
| (2:1) | | | | | | | | |
| 11.4/1.75 | 3.3 | 12.7 | 11.3 | 3.6 | 1.0 | 2.1 | 50.3 | 69.0 |
| (4:1) | | | | | | | | |

*Temp: 221° C.

Both the loading of Mo, Co and the ratio of Mo/Co influence the performance of catalyst. The fact that the catalyst without either Mo or Co was not active at the conditions applied, indicates that some alloy of Mo and Co is formed on the surface of the substrate and is likely the active catalytic state of the supported metals.

5. Effect of Cs Loading on the Performance of Mo/Co/C_{S}/al₂O₃ to Alcohol Synthesis from Syngas

Catalysts used: Mo, Co loading are 8.5 wt %, 3.5 wt % to the substrate.

Cs loading varies from 0-3.6 wt % to the substrate.

Test Conditions: Test Temp: 237-250° C. Pressure: 1200 psig. GHSV: 6000 h⁻¹

Syngas: CO/H₂=1:1

Test Time: 5 hours

TABLE 5
Effect of Cs loading on the performance of
Mo/Co/Cₛ/Al₂O₃ to alcohol synthesis from Syngas

| Cs loading | | | | | | | | |
|---|---|---|---|---|---|---|---|---|
| Wt % | Con. wt | | | | | | Alcohol | |
| (molar ratio | % of | Selectivities of Alcohols C Mol % | | | | | Productivity · | G |
| Co:Cs | CO | MeOH | ETOH | 1-PrOH | 1-BuOH | Other-ROH | g/kgcat · h | Gallon/BDT |
| 0 | 3.14 | 17.9 | 13.2 | 5.72 | 1.99 | 1.75 | 85.0 | 89.4 |
| 0.73 | 3.44 | 20.8 | 16.3 | 6.57 | 2.39 | 2.71 | 108.9 | 106.4 |
| (1:0.0.10) | | | | | | | | |
| 1.39 | 3.22 | 18.9 | 14.7 | 6.47 | 2.38 | 2.99 | 92.8 | 97.6 |
| (1:0.17 | | | | | | | | |
| 2.20 | 2.69 | 19.6 | 15.7 | 7.03 | 3.19 | 3.25 | 83.4 | 105 |
| (1:0.28) | | | | | | | | |
| 2.90 | 4.10 | 15.7 | 14.5 | 6.42 | 2.70 | 2.88 | 103.5 | 89.8 |
| (1:0.37) | | | | | | | | |
| 3.60 | 3.93 | 14.4 | 14.3 | 6.42 | 2.71 | 3.04 | 94.4 | 85.8 |
| (1:0.46) | | | | | | | | |

The selectivity of the reaction to alcohols increases with the loading of Cs and is optimized at Cs loading of 0.73-2.2%. Continuing to increase Cs loading beyond these levels will decrease the selectivity and the yield of alcohol. G value is 97.6-106.6 gallon/BDT of biomass when the Cs loading is 0.73-2.2%.

6. Distribution of alcohols obtained as a function of Cs loading

Table 6 shows the distribution of alcohols obtained from the experiments shown above in Table 5.

TABLE 6
Distribution of alcohols obtained as a function of Cs loading

| Cs Loading % | 0 | 0.73 | 1.39 | 2.2 | 2.9 | 3.6 |
|---|---|---|---|---|---|---|
| Methyl Alcohol | 54.24% | 52.60% | 51.28% | 50.24% | 47.17% | 45.18% |
| Ethyl alcohol | 28.69% | 29.61% | 29.08% | 29.04% | 31.25% | 32.39% |
| 1-Propanol | 10.79% | 10.41% | 11.17% | 11.27% | 12.03% | 12.13% |
| 1-Butanol | 3.28% | 3.50% | 3.80% | 4.74% | 4.68% | 4.94% |
| 1-Pentanol | 1.17% | 1.40% | 1.68% | 1.96% | 1.98% | 2.17% |
| 1-Hexanol | 0.53% | 0.79% | 0.88% | 0.80% | 0.89% | 1.27% |
| 2-propanol | 0.72% | 0.70% | 0.89% | 0.60% | 0.76% | 0.76% |
| 2-butanol | 0.21% | 0.30% | 0.33% | 0.15% | 0.27% | 0.32% |
| 2-methyl-1-propanol | 0.38% | 0.58% | 0.73% | 1.19% | 0.87% | 0.70% |
| 2-Pentanol | 0.0% | 1.13% | 0.16% | 0.00% | 0.12% | 0.14% |
| Total | 100.00% | 100.00% | 100.00% | 100.00% | 100.00% | 100.00% |
| VM $/gallon | 1.59 | 1.67 | 1.67 | 1.71 | 1.68 | 1.52 |

The methanol selectivity decreases with the increase of Cs loading, but ethanol and other high alcohols increase with the loading of Cs. This indicated that basic promoters will increase the selectivity of higher alcohols. Combining the selectivity and alcohol distribution in the liquid, the highest variable margin (VM) is $1.71 per gallon when the Cs loading is at 2.2 wt %. Variable margin is the difference between the raw material cost and the selling price of the alcohols produced. The following selling prices were used in the weighted average sales price calculations: methanol $1.50/gal., ethanol $2.30/gal., propanol and higher alcohols at $3.00/gal. The raw material cost used assumes the thermal conversion of biomass to syngas containing hydrogen and carbon monoxide, at a price for biomass of $35 per bone dry ton.

Of course it is understood that the foregoing are preferred embodiments of the invention, and that various changes and alterations can be made within the scope of the following claims, as interpreted and applied in accordance with the principles of patent law, including the Doctrine of Equivalents.

1. A carried catalyst comprising:
elemental molybdenum, cobalt or their alloy and an alkali or alkaline earth metal, and/or hydrides thereof, in an elemental ratio of about 2-1:1:0.08-0.30, carried on a porous, inert particularized material.

2. The carried catalyst of claim 1 in which the elemental ratio is about 1.5:1:0.26-0.28.

3. The carried catalyst of claim 2 in which the substrate is one of particularized carbon, titanium dioxide, zirconium dioxide and alumina.

4. The carried catalyst of claim 3 in which the substrate is alumina (Al₂O₃).

5. The carried catalyst of claim 4 in which the alumina is in spherical particle form, having a particle size of from about 1.5 to about 2.0 millimeters (mean diameter), a density of about 0.63 grams per cubic millimeter, a surface area of about 210 m² per gram, and a pore volume of about 0.75 cubic millimeters per gram.

6. The carried catalyst of claim 5 in which the particle size of the alumina is about 1.8 millimeters.

7. The carried catalyst of claim 1 in which the alkali or alkaline earth metal is cesium.

8. The carried catalyst of claim 1 comprising from about 5.7 to about 11.4 wt % Mo carried on said substrate.

9. The carried catalyst of claim 8 comprising from about 1.75 to about 3.5 wt % of Co carried on said substrate.

10. The carried catalyst of claim 1 comprising from about 8.5 to about 10 wt/wt % molybdenum carried on said carrier.

11. The carried catalyst of claim 1 comprising Cs loaded onto said substrate at from about 0.73 to about 2.9 wt % to the carrier.

12. The carried catalyst of claim 1 comprising Cs loaded onto said substrate at from about 0.73 to about 2.2 wt % to the carrier.

13. A precursor for a carried catalyst comprising:
the salts or oxides of molybdenum, cobalt and an alkali or alkaline earth metal promoter, carried on a porous, inert particularized material in an elemental Mo to Co to alkali or alkaline earth metal ratio of about 2-1:1:0.08-0.30.

14. A method for making a carried catalyst comprising:
heating a porous, inert particularized material carrying the salts or oxides of molybdenum, cobalt and an alkali or alkaline earth metal promoter, carried in an elemental Mo to Co to alkali or alkaline earth metal ratio of about 2-1:1:0.08-0.30, to a temperature of about 600° C. to about 900° C. for about 3 to about 7 hours in a reducing atmosphere.

15. A method for making a carried catalyst comprising:
impregnating a porous, inert particularized material with a salt of molybdenum, a salt of cobalt and a salt of an alkali or alkaline earth metal promoter, carried in an elemental Mo to Co to alkali or alkaline earth metal ratio of about 2-1:1:0.08-0.30; calcining at least the impregnated salts of Mo and Co, and heating the resulting material to a temperature of about 600° C. to about 900° C. for about 3 to about 10 hours in a reducing atmosphere.

16. A method for making alcohols from a gas comprising hydrogen and carbon monoxide comprising:
passing the gas through a reactor containing a carried catalyst comprising elemental molybdenum, cobalt and an alkali or alkaline earth metal, and/or hydrides thereof, in an elemental ratio of about 2-1:1:0.08-0.30, carried on a porous, inert particularized material, at a temperature of from about 240 to about 270° C., a pressure of from about 1000 to about 1200 psig, and a Gas Hourly Space Velocity of from about 4000 to about 6000 h⁻¹.

17. The carried catalyst of claim 9 comprising Cs loaded onto said substrate at from about 0.73 to about 2.9 wt % to the carrier.

18. The carried catalyst of claim 9 comprising Cs loaded onto said substrate at from about 0.73 to about 2.2 wt % to the carrier.
